(19) United States
(12) Patent Application Publication
(10) Pub. No.: US 2010/0261953 A1
(43) Pub. Date: Oct. 14, 2010

(54) APPARATUS AND METHOD FOR PELVIC FLOOR REPAIR IN THE HUMAN FEMALE

(75) Inventors: Philip A. Townsend, Brooksville, FL (US); Michael Afremov, St Louis Park, MN (US); Roderick B. Brown, Glenwood, MN (US)

Correspondence Address: NIKOLAI & MERSEREAU, P.A., 900 SECOND AVENUE SOUTH, SUITE 820, MINNEAPOLIS, MN, 55402, US

(73) Assignee: Minnesota Medical Development, Inc., Plymouth, MN (US)

(21) Appl. No.: 12/421,116

(22) Filed: Apr. 9, 2009

(51) Int. Cl. A61F 2/00

(52) U.S. Cl. 600/37

(57) ABSTRACT

A prosthesis for addressing pelvic organ prolapse in females comprises a frame fabricated from a shape memory material that supports a thin, flexible sheet in a stretched condition when the frame is unconstrained. The frame is shaped so as to conform to and be supported by bone structures and muscle tissue in the pelvic basin while providing needed support to pelvic organs to maintain them in a proper position. The use of a shape memory material allows the prosthesis to be rolled or folded into a reduced size for ease of placement through a small incision in the wall of the vagina, but that springs back to its memorized shape following deployment from a delivery sheath.

### BACKGROUND OF THE INVENTION

I. Field of the Invention

This invention relates generally to a surgically implantable prosthesis for hernia repair, and more particularly to an implantable device especially designed for pelvic floor repair.

II. Discussion of the Prior Art

The following definitions apply to terminology used in the present specification and claims:

Genital prolapse or pelvic organ prolapse refers to a loss of fibromuscular support of the pelvic viscera that results in vaginal protrusion. The prolapse is usually described according to the area of the vagina in which it occurs.

An anterior vaginal prolapse generally involves the bladder (cystocele), and often involves hypermobility of the urethrovesical junction as well (cystourethrocele).

A posterior vaginal prolapse often involves protrusion of the rectum into the vaginal canal (rectocele) and/or protrusion of a loop of small bowel in a peritoneal sac (enterocele).

Procidentia refers to a complete protrusion of the uterus and vagina.

The term vaginal vault prolapse refers to a complete or partial inversion of the vaginal apex, most commonly occurring in patients who have had a hysterectomy.

The term pseudorectocele describes an inadequate or defective perineum resulting in exposure of the mid-portion of the posterior vaginal wall. It mimics the appearance of a rectocele, but does not involve creation of a rectal pouch that incorporates both rectal and vaginal walls with loss of vaginal rugation.

An enterocele is the herniation of a peritoneal sac (usually filled with small bowel) through the vaginal apex. An enterocele may be further classified as a traction enterocele or a pulsion enterocele.

A traction enterocele is a protrusion of the posterior cul-de-sac that is pulled down by the prolapsing cervix or vaginal cuff.

A pulsion enterocele is a protrusion of the cul-de-sac through the vagina resulting from chronically increased intra-abdominal pressure. Pulsion enteroceles are frequently large and always contains small bowel. Enteroceles are usually encountered as a dissect through the rectal-vaginal septum, but may also occur in the space between the bladder and the anterior vaginal wall.

Factors which predispose women of all ethnic groups to the development of prolapse include vaginal delivery, chronic increases in intra-abdominal pressure, obesity, advancing age and estrogen deficiency. Pelvic trauma and pelvic surgery may damage the neuromuscular structures, connective tissue and muscles of the pelvic floor, and vaginal delivery leads to stretching, dislocation, tearing and avulsion of pelvic tissues. Neurological injury to the pudendal nerve may also occur, as has been demonstrated in women with stress incontinence and pelvic organ prolapse. Chronic straining, as through heavy lifting, may also damage the pudendal nerve and lead to subsequent pelvic floor dysfunction by compromising neuromuscular function.

Post-hysterectomy vaginal vault prolapse is a distressing and increasingly common problem. It may occur following vaginal or abdominal hysterectomy and often results from inattention to the proper reconstruction of vaginal apex support following removal of the uterus.

Pelvic organ prolapse can present many symptoms, depending on the organs involved. The most frequent symptom is a complaint of a protrusion or bulge from the vagina that worsens with prolonged standing or walking. In some cases, the prolapse may be large enough to impair ambulation. Other common symptoms include low back pain, urinary incontinence, voiding difficulty and difficulty emptying the rectum. Changes in the vaginal epithelium are frequently present in women with prolapse. In younger women, the vaginal skin may be hypertrophic, but in older women it will be atrophic, particularly if they are not receiving estrogen replacement therapy. Sexual dysfunction may also be present in women with prolapse due to alterations in vaginal anatomy and pelvic organ function.

In the human female, the endopelvic fascia, attaching the bladder, uterus, vagina and rectum to the pelvic sidewalls, is a fibrous connective tissue layer extending diffusely throughout the pelvic floor to form a continuous sheet-like mesentery. It is subdivided into the parametrium and paracolpium. The parametrium consists of the cardinal and uterosacral ligaments, which provide part of the structural support of the uterus. These so-called “ligaments” are really only two different parts of a single mass of loose tissue. The paracolpium attaches the upper two-thirds of the vagina to the pelvic wall and is continuous with the parametrium when the uterus is in situ. It helps suspend the vaginal apex after hysterectomy.

The vagina has three main levels of support:

Level I support includes the vagina apex and the paracervical vagina, which is suspended by the long connective tissue fibers of the superior paracolpium.

The mid-portion of the vagina (Level II) is attached laterally, stretching between the bladder and the rectum and supported by the inferior portion of the paracolpium. At this level, the anterior vaginal wall and the endopelvic fascia merge to form the pubocervical fascia, which underlies the bladder. Posteriorly, the endopelvic fascia merges with the posterior vaginal wall to form the rectovaginal fascia. This layer prevents the rectum from protruding through the posterior vaginal wall.

The lowest portion of the vagina (level III) is found at the vaginal introitus and has no intervening paracolpium to suspend it. At this level, the vagina fuses directly with the levator ani muscles laterally, the urethra anteriorly and the perineum posteriorly.

Injury to the suspensory fibers at level I may result in vaginal and uterine prolapse and enterocele formation. Damage to the pubocervical fascia or rectovaginal fascia (the supportive fibers of level II) leads to the development of cystocele and rectocele, respectively. Injury often occurs at both levels and results in a combination of defects.

Another important component of the pelvic floor is the levator ani muscles, which may be subdivided into pubococcygeal or “pubovisceral” portion and an iliococcygeal portion.

The levator ani muscles are critical in pelvic floor support. These muscles maintain a constant basal tone that maintains the uterus and vagina in place. Above the levator ani, the ligaments and fascia stabilize the organs in position. Constant adjustments in muscular activity prevent the stretching of the pelvic ligaments. Contraction of the pubovisceral muscle pulls the rectum toward the pubic bone, closing the urogenital hiatus and compressing the urethra, vagina and uterus. The pelvic floor should be seen as a dynamic trampoline that is constantly expanding and contracting in response to changing stimuli rather than a static slab. The levator muscles contract reflexively during periods of increased intra abdominal pressure (coughing, sneezing, etc.). In this process, the urethra, vagina and rectum are compressed against the levator plate, maintaining their normal positions in the pelvis. Any stretching or laceration of the levator muscles or endopelvic fascia can result in widening of the urogenital hiatus and a rotation in the axis of the levator plate with the subsequent development of a predisposition to uterine or vaginal prolapse.

The first meaningful advance in the treatment of pelvic floor prolapse was the development of pessaries that functioned as trusses. Generally speaking, a pessary is a device that can be inserted into the vagina to support sagging organs. Their use gained considerable popularity in the mid-19^{th} century. During the 20^{th} century, advances in the understanding and surgical treatment of pelvic floor prolapse progressed at an increasing rate, particularly during the first, middle and last decades. In 1909, Dr. George White of Georgia was one of the first to report a cystocele repair using a transvaginal paravaginal approach. His correct assessment of the importance of what is now referred to as “level II” pelvic organ support by the attachment of the pubocervical fascia to the Arcus Tendineous of the pelvic sidewalls was rediscovered by mainstream workers in the field in the 1950's. This procedure involves difficult and specialized suturing techniques.

In the 1950's Dr. Milton McCall of Louisiana emphasized the importance of uterosacral ligaments in the so-called “level I” support of the vaginal vault. Such support was reconstituted by him at a time of hysterectomy with his development of a culdoplasty technique that included suture reattachment of the severed uterosacral ligaments to the vaginal vault to prevent subsequent pelvic floor herniation in the form of enterocele and vaginal vault prolapse.

More recently, in the 1990's, emphasis has been placed on the hernia nature of prolapse, leading to a change from absorbable suture material to permanent suture. Again, in the 1990's, pelvic anatomist, John DeLancey of Michigan, published a Biomechanical Analysis of Normal Vaginal Anatomy. This work identified specific surgical goals for each of the three levels of support. These are proximal vaginal suspension (level I support), mid-vaginal lateral attachment (level II support) and distal vaginal fusion to the perineum and urogenital fascia. These are the basic concepts that contemporary pelvic surgeons must satisfy to complete a pelvic herniation surgery.

For much of the 20^{th} century, surgical repair of pelvic floor hernias was based upon the assumption by the influential pelvic surgeon, Howard Kelly of Johns Hopkins Hospital, and other workers in the field, that fascial attenuations of the vaginal walls were the cause of these hernias. Mid-line, anterior and posterior vaginal wall fascial plication, otherwise known as anterior and posterior colporrhaphy, respectively, generally with absorbable suture material, was the mainstay of surgical treatment for most of these hernias.

The high recurrent herniation rates, particularly that of cystocele formation with this approach, led to intensive clinical research into the exact defects involved in the pelvic floor hernia formation. These defects were considered by these researchers to be due to injuries sustained during childbirth and to be specific in site as opposed to simple attenuation. Such anatomic site specific damage lends itself to the concept of pelvic reconstructive surgery. Dr. A. Colin Richardson of Georgia classified damage to the pubocervical fascia between the bladder and anterior wall as proximal, distal, central and lateral. Other workers, such as Dr. David Nichols of Rhode Island, encouraged gynecologists to both identify and repair each of these defects and to return support attachments to their original anatomic location. This includes, for example, repair of a paravaginal hernia by reattaching with suture the pubocervical fascia to the Arcus Tendineus. Such pelvic reconstructive surgery is heavily dependent for success upon the training, skill and expertise of individual surgeons. It frequently demands relatively long operative times.

Thus a need exists for a surgically implantable device that will rely less upon the attributes of individual surgeons for success and that will involve a shorter operating time.

Furthermore, a need exists for a surgically implantable device having the ability to repair damage, and thus restore normal function, to crucial level II supporting mechanisms without the need for difficult and specialized suturing techniques.

A further need exists for a surgically implantable prosthesis having the ability to restore level I support that can be rapidly positioned and held in place with a minimum of suturing.

### SUMMARY OF THE INVENTION

The foregoing needs are satisfied by the present invention that relates to an implant for pelvic floor repair. The implantable prosthesis consists of an expandable frame for holding open a sheet of a suitable biological graft or a synthetic mesh. The device is designed to be held in place in the pelvis by low level recoil forces imposed between the device frame and the pelvic walls. With regard to anterior pelvic floor repair, such recoil forces include, but are not limited to, those between the frame and the fibromuscular pelvic sidewalls in close proximity to the so-called “plane of maximum dimension”. Anatomical structures on each side of the pelvis known as the Arcus Tendineous Fascia Pelvis laterally, the Sacrospinous Ligament posteriorly and the Inferior Pubic Ramus anteriorly will be in close proximity to the plane of the frame.

Broadly stated, the implantable device of the present invention may comprise a sheet of mesh fabric or graft material of a predetermined shape configuration along with a support frame for maintaining the sheet in its predetermined shape configuration following implantation of the device proximate the pelvic floor of a female patient. The support frame is affixed to the sheet of mesh fabric or graft material and includes first and second wing portions that are bilaterally symmetrical about a central axis of the device. The wing portions include rounded wing tip portions at first ends thereof that are adapted to abut the pelvic wall in proximity to the Sacrospinous Ligaments, when implanted in a female patient, said wing tip portions on the first and second wing portions being integrally joined to one another by a concave, arcuate segment. End portions of the first and second wing portions that are opposite to the wing tip portions are dimensioned to rest upon the posterior surface of the pubic rami and/or Symphysis Pubis of said female when the wing tip portions engage the patient's posterior pelvic wall proximate to the sacrospinous ligament.

The frame itself is preferably formed from a biodegradable polymer exhibiting shape memory properties but may also comprise a plurality of strands of Nitinol wound as a cable.

Because of the shape memory property of the frame, it is capable of being rolled or otherwise folded into a tubular configuration of a relatively small radial dimension for delivery through a surgical incision through the vaginal wall in its low profile configuration, but once inside the body, proximate the pelvic floor, will unfurl to its predetermined desired shape.

### DESCRIPTION OF THE DRAWINGS

The foregoing features, objects and advantages of the invention will become apparent to those skilled in the art from the following detailed description of a preferred embodiment, especially when considered in conjunction with the accompanying drawings in which:

FIG. 1 is a plan view of a first embodiment when in an expanded, unconstrained condition;

FIG. 2 shows the device of FIG. 1 in a rolled, low profile condition adapted to be delivered into the pelvic compartment via a vaginal incision of approximately 3 cm in length;

FIG. 3 is a superior view of the female pelvic diaphragm showing placement of the device of the present invention in treating pelvic floor herniation;

FIG. 4 is a plan view of an alternative embodiment when in an expanded, unconstrained condition;

FIG. 5 is a plan view of a further alternative embodiment when in an expanded unconstrained condition;

FIG. 6 is a cross-sectioned view taken through the frame of the embodiment of FIG. 5;

FIG. 7 is an anatomical skeletal drawing illustrating an anterior view of the female pelvis showing placement of the device of FIG. 4 in treating pelvic organ prolapse;

FIG. 8 is a sagittal section view showing approximate placement of the prosthesis of the present invention for addressing pelvic organ prolapse;

FIG. 9 is a schematic saggital view illustrating cystocele; and

FIG. 10 is a view like FIG. 9 but showing placement of the prosthesis in treatment of cystocele.

### DESCRIPTION OF THE PREFERRED EMBODIMENT

This description of the preferred embodiments is intended to be read in connection with the accompanying drawings, which are to be considered part of the entire written description of this invention. In the description, relative terms such as “lower”, “upper”, “horizontal”, “vertical”, “above”, “below”, “up”, “down”, “top” and “bottom” as well as derivatives thereof (e.g., “horizontally”, “downwardly”, “upwardly”, etc.) should be construed to refer to the orientation as then described or as shown in the drawings under discussion. These relative terms are for convenience of description and do not require that the apparatus be constructed or operated in a particular orientation. Terms such as “connected”, “connecting”, “attached”, “attaching”, “join” and “joining” are used interchangeably and refer to one structure or surface being secured to another structure or surface or integrally fabricated in one piece, unless expressively described otherwise.

Referring first to FIG. 1, it shows a plan view of the pelvic floor repair patch 10 constructed in accordance with a first embodiment of the present invention. It is seen to comprise a sheet of mesh fabric 12 having a predetermined shape configuration. Without limitation, the sheet of mesh fabric may be formed from polypropylene or PTFE, both of which have been used in the past in constructing implantable medical prostheses. While such mesh fabrics are preferred, it is also contemplated that the sheet 12 may comprise a xenograft, such as appropriately treated porcine dermis tissue.

The sheet material 12 is provided with a support frame 14 for maintaining the sheet 12 in its predetermined shape configuration following placement of the patch 10 proximate the pelvic floor of a female patient.

As seen in FIG. 1, the support frame 14 used in this embodiment is affixed to the sheet 12, such as by closely spaced stitches 16. The support frame 14 comprises first and second wing portions 18 and 20 that are preferably bilaterally symmetrical about an imaginary central axis 21 of the device. Each of the wing portions 18 and 20 includes rounded wing tip portions 22 and 24 at first ends thereof and these wing tip portions on the first and second wing portions 18 and 20 are integrally joined to one another by a concave, arcuate segment 26.

In the embodiment of FIG. 1, the ends of the wing portions 18 and 20 opposite the wing tip portions 22 and 24 are generally rounded as at 28 and 30. Convex arcuate segments 29 and 31 join the wing tips 22 and 24 to their respective opposite ends 28 and 30.

The support frame 14 may comprise one or more strands of a shape memory material, multiple strands will be wound together as a cable. Without limitation, the strands may be made from a shape memory metal, such as Nitinol, or alternatively, from a suitable biodegradable polymer having elastic properties.

The particular polymer to be used as a biomaterial in forming the frame is one that will match the mechanical properties and the time of degradation to the needs of the application. The ideal polymer for this application will not evoke an inflammatory/toxic response, is able to be metabolized in the body after fulfilling its purpose and one that leaves no significant trace, is sterilizable and can readily be processed into the desired configuration.

Polydioxanone is a bio-degradable polymer having a glass transition temperature in a range of from −10° C. and 0° C. and a crystallinity of about 55%. The presence of an ether oxygen group into the backbone of the polymer chain gives the material a good flexibility. It also exhibits a shape memory property. A monofilament of polydioxanone loses about 50% of its initial breaking strength after three weeks and is absorbed within six months. This provides ample time for tissue ingrowth into the mesh to take place.

As further seen in FIG. 17 the sheet 12 generally follows the contour of the frame member, but with the border of the sheet material 12 extending laterally beyond the support frame.

To prevent unraveling of the multiple strands comprising the cable frame 14, it has been found expedient to apply a tubular ferrule 32 to the free ends of the strands to form a closed loop. Where the frame comprises multiple strands of Nitinol wire twisted together as a cable, the ferrule 32 may be laser beam welded in place surrounding the opposed ends of the strands. Where the frame comprises a polymer, the free ends may be fused together by melting and then allowed to solidify. In either case, fraying of the multiple strands is prevented.

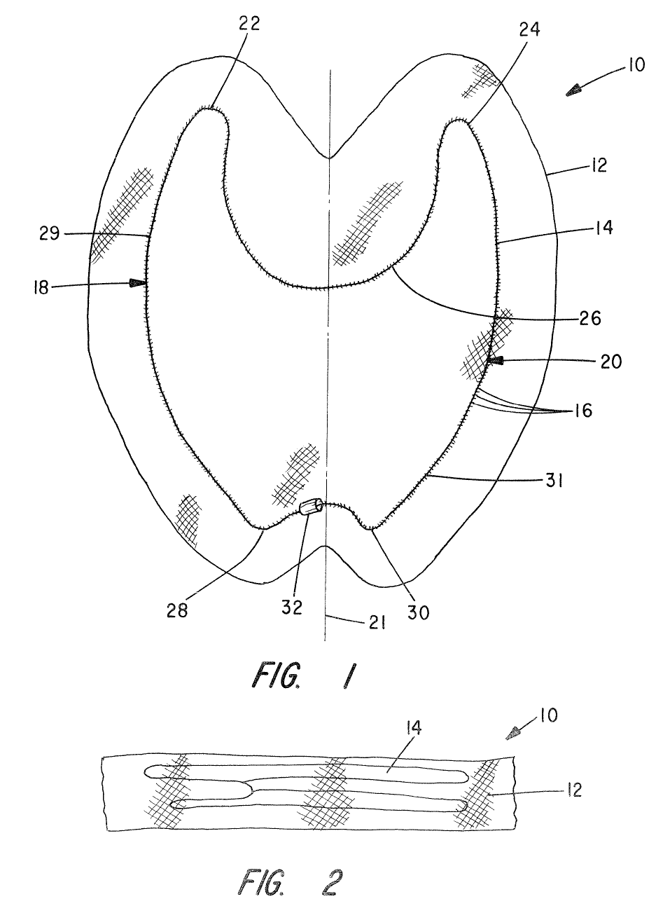

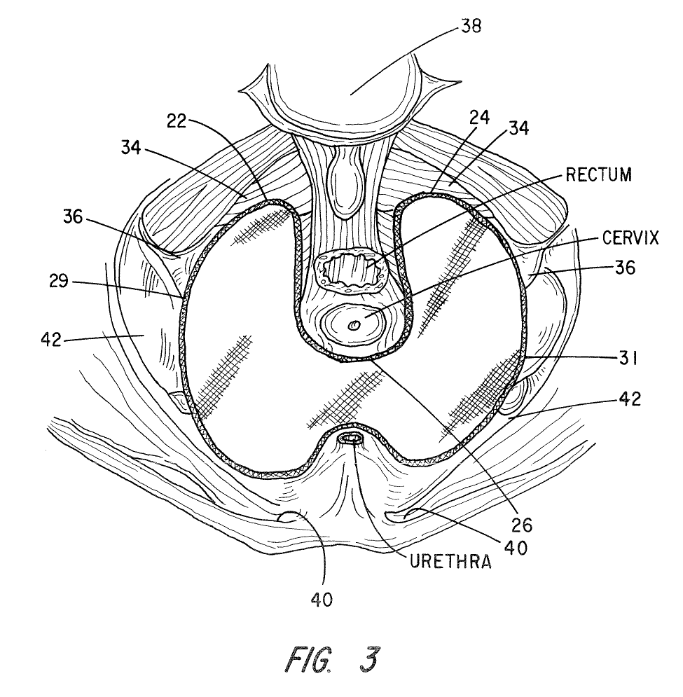

With the frame 14 being fabricated from an elastic material, it is possible to roll up the device from the configuration shown in FIG. 1, which is generally planar, to a tubular configuration as shown in FIG. 2. As such, the device may then be inserted through an incision 2-3 cm in length in the wall of the vagina into the pelvic cavity where it is allowed to unfurl by elastic recoil and thus reassume the shape configuration shown in FIG. 1. The surgeon may then use his or her fingers to position the device in the appropriate pelvic plane described previously to best address the type and degree of organ prolapse that the surgery is intended to correct. Because of the inherent property of the frame, it reduces bunching or crinkling of the mesh due to uneven suturing of prior art patch materials used in pelvic floor repair. Such bunching or crinkling commonly results in dyspareunia during coitus.

If it is desired to remove a metal frame 14 following placement of the sheet 12 and before closing the incision in the vaginal wall, the sheet may be formed so as to include a plurality of spaced-apart “belt-loop” like appendages thereon through which the frame 14 is strung. After being appropriately spaced, bio-degradable anchoring tacks can be used to hold the sheet 12 in place, the frame 14 can be stripped out from the belt loop appendages and removed from the patient. Of course, if the frame 14 comprises a biodegradable polymer, there is no need to remove it because it will be absorbed by the body following tissue ingrowth through the sheet material during the period of three months or so post-surgery.

Referring next to FIG. 3, there is shown a superior view of the female pelvic diaphragm showing placement of the device of the present invention in treating pelvic floor herniation. When appropriately placed, the rounded wing tip portions 22 and 24 on the prosthesis frame 14 are arranged to rest against the pelvic wall in the region of the Sacrospinous Ligament 34 that extends between the sacral spine 36 and the sacrum 38. The opposite end portions 28 and 30 yieldingly engage the region of the lower symphysis and adjacent inferior pubic rami 40. When the device is so positioned, the convex arcuate portions of the frame 29 and 31 will be supported by the pelvic sidewalls in the region of the Arcus Tendineous Fasciae Pelvis 42. This placement results in the anterior end portion of the concave arc segment 26 looping around the vagina at the level of the cervix, C. The concave segment connecting ends 28 and 30 provides clearance for the urethra, U.

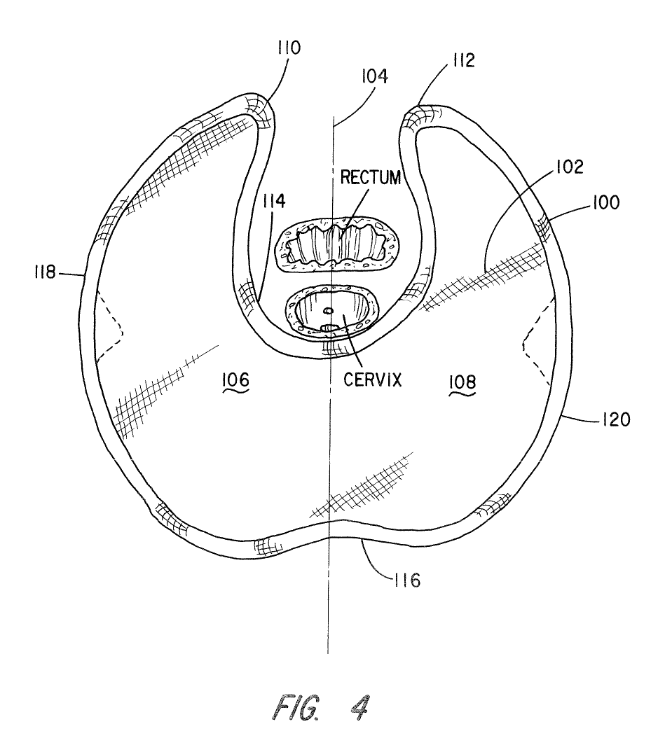

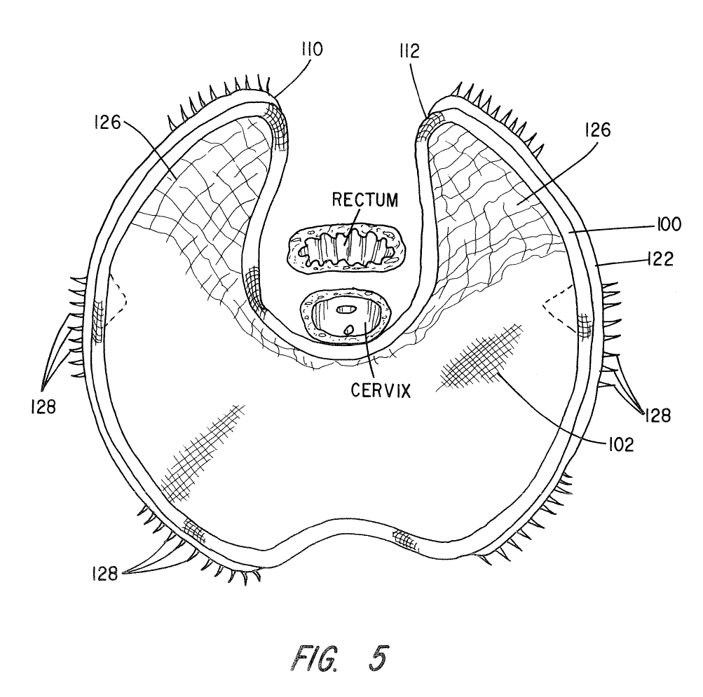

FIG. 4 is an alternative embodiment of the device for addressing repair of Level I and Level II support. It comprises a frame member 100 supporting a biocompatible sheet, such as a polypropylene mesh or a treated porcine dermis material 102.

As in the embodiment of FIG. 1, the frame is again bilaterally symmetrical about an imaginary central axis 104. It is comprised of a plurality of generally circular arcs that are integrally joined to form a pair of wing-shaped members 106 and 108 on opposite sides of the axis 104.

The arcs define wingtip portions 110 and 112 that when placed in a female patient are arranged to abut the region of the sacrospinous ligaments. These wingtip portions are joined to one another by a concave, arcuate segment that is sized and shaped so as not to interfere with the rectum, R, and providing support to the vagina at the level of the cervix, C. The concave, arcuate portion 116 allows engagement of the implant with the lower Symphysis Pubis inferior pelvic rami. The convex arcuate segments 118 and 120 are designed such that they resiliently engage the pelvic sidewalls in a plane located slightly above the iscial spine, which is proximate the pelvic plane of the greatest dimensions.

In the embodiment of FIG. 4, the frame 100 is preferably molded from a biocompatible, biodegradable polymer exhibiting shape memory properties. Polyurethanes formed from a high molecular weight poly(e-caprolactone) and a high weight fraction of hard-segment-determining blocks exhibit a high shape-memory property. Block copolymers made with polyethylene terephthalate and polyethyele oxide is also a potential candidate as are copolymers of polyglycolide (PGA) and polylactide (PLA). Another potential candidate for the frame material is a polymer called polynorbornene. Readers desiring additional information on shape memory polymers exhibiting biodegradable properties are referred to an article entitled “Shape-Memory Polymers” authored by Andreas Lendlein and Steffen Kelch, Angew. Chem. Int. Ed. 2002, 41, pages 2034-2057, the contents of which are hereby incorporated by reference.

Turning next to FIG. 5, there is shown a further embodiment in which the sheet 102 has fibers interwoven in the mesh so as to stimulate tissue ingrowth when the prosthesis is to be used for repairing Level I as well as Level II vaginal support. In the area of the wingtips 110 and 112, the mesh sheet 102 is interwoven with fibers of polyethylene terephthalate (PET) as identified by numeral 126, a material known to induce fibrosis, whereby the mesh sheet 102 becomes secured prior to the loss of resiliency in the frame due to biodegradation with time.

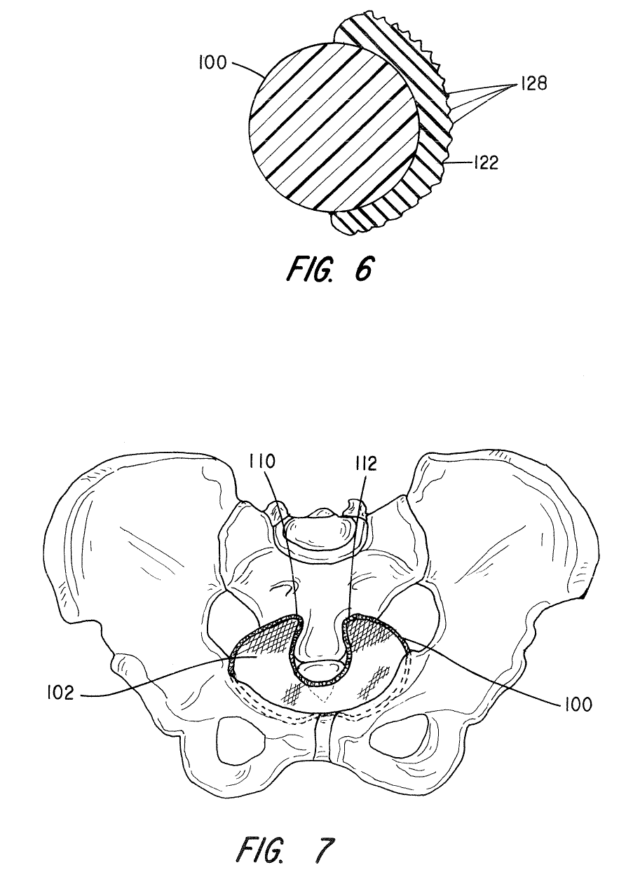

As a further option, to reduce the possibility of patient discomfort due to pressure of the resilient frame with pelvic tissue prior to its being absorbed, the frame may be formed in a molding operation to exhibit a cross section such as depicted in FIG. 6 hereof. The polymer frame member 100 is integrally molded to exhibit a cushioning layer 122 formed of a soft, deformable foam material. The cushioning layer 122 need only span the arcuate portions 118 and 120 of the frame member 100. The cushioning layer 122 is sufficiently resilient that it can deform to spread the contact force over a greater area, thereby reducing the contact pressure between the frame structure and the tissue that it abuts. The embodiment of FIG. 5 and the cross-section of FIG. 6 also show that the cushioning layer 122 may have raised tread-like projections as at 128 extending radially from the surface thereof which aid in fixing the frame in fibromuscular tissue of the pelvic side walls. These projections may be integrally molded with the cushion layer 122.

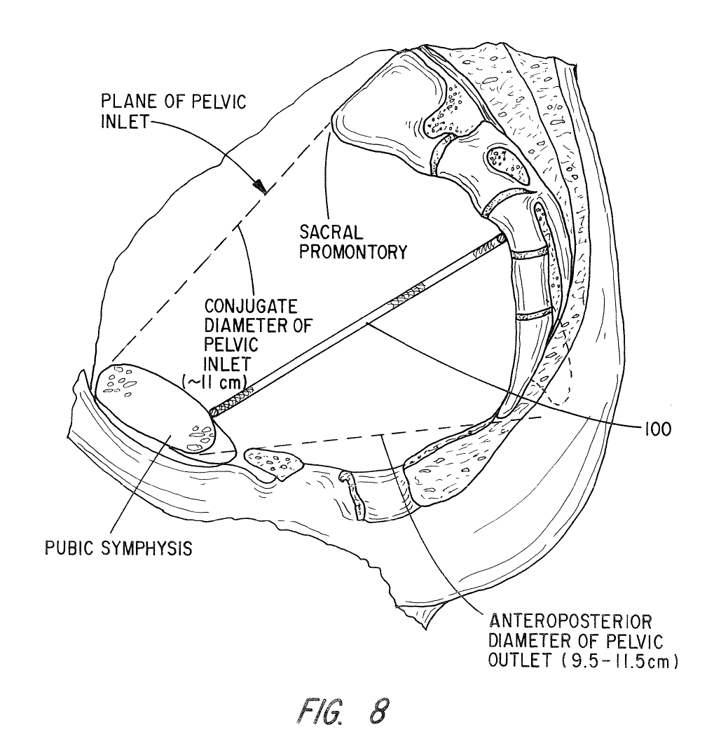

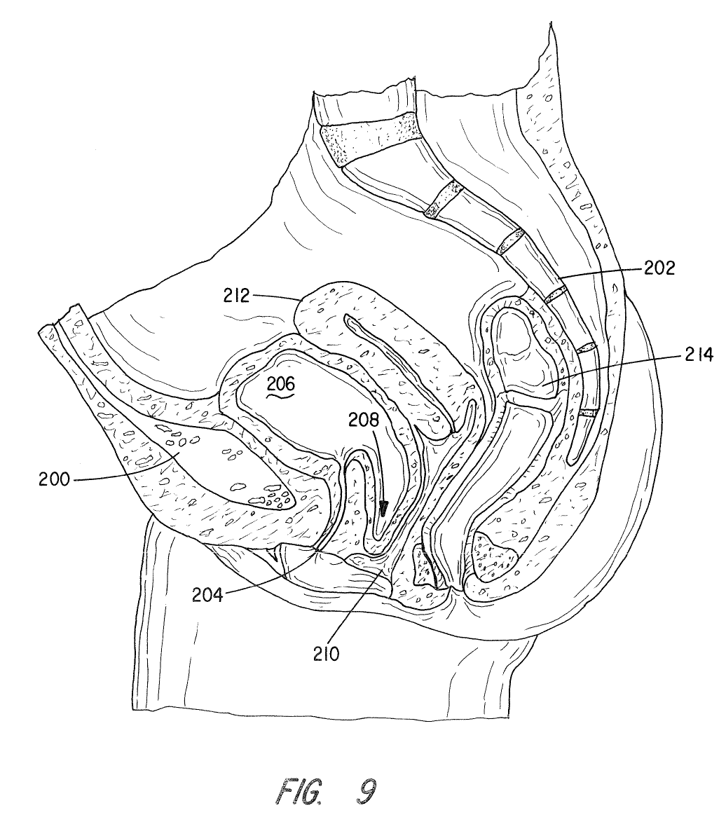

FIG. 7 is an anterior view of the skeletal female pelvis on which the prosthesis of the present invention has been added to generally illustrate the placement of the prosthesis when addressing forms of pelvic organ prolapse. The anterior portion of the frame 100 is made to engage the inferior pubic symphysis as best seen in FIG. 8 while the wingtip portions 110 and 112 thereof abut the Sacrospinous Ligament proximate the joint between the third and fourth sacral segments. As the name suggests, the Sacrospinous Ligament is a thin, triangular ligament attached by its apex to the ischial spine, and medially, by its broad base, to the lateral margins of the sacrum and coccyx. When the prosthesis is so positioned, the convex arcuate segments 118 and 120 of the prosthesis are somewhat elevated relative to the ischial spine and engage the region of the Arcus Tendineous Fascia Pelvis.

FIG. 9 is a schematic illustration of the condition known as cystocele in which the posterior wall of the bladder prolapses into the vaginal space due to a defect in the anterior vaginal wall fascia. In FIG. 9, the pubic symphysis is identified by numeral 200 and the ischial spine by numeral 202. The urethra 204 leads to the urinary bladder 206 exhibiting a cystocele 208 or protrusion into the vaginal canal 210 leading to the uterus 212. The rectum is identified by numeral 214.

The bladder and urethra are separated from the vagina by the pubocervical fascia. Intact fascia prevents the bladder from bulging down into the vagina. Females with cystocele have a defect or weakness in this fascia.

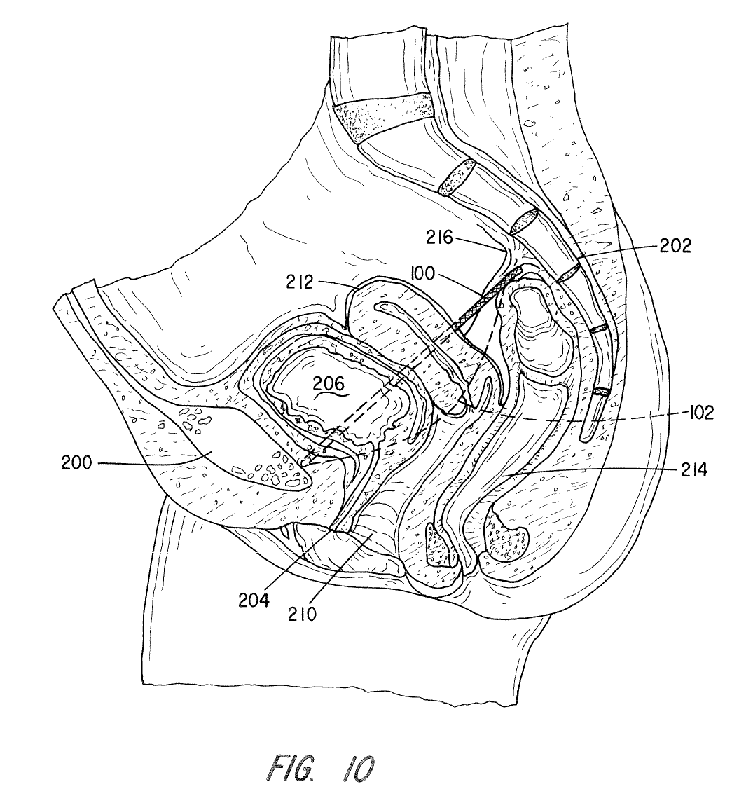

FIG. 10 is a view like that of FIG. 9 but with the prosthesis of the present invention deployed as previously described so as to provide lateral support to the bladder 206 and repairing the cystocele. In this view, the cross-section of the frame 100 is displayed with its anterior portion engaged with the pubic symphysis or inferior rami and its posterior wingtip portions abutting the region of the sacrospinous ligaments identified in FIG. 10 by numeral 216.

A method for the surgical repair of anterior vaginal wall prolapse, or cystocele, is described with reference to FIGS. 7 through 10. The surgical procedure involved will, in its general description, be well recognized by workers in the field. A concomitant procedure for stress urinary incontinence (SUI), both occult and overt, may be carried out under the same anesthetic.

After standard preoperative preparation of the patient has been completed in an optimal manner, she will receive appropriate anesthesia and be placed in the so called modified lithotomy position. She will then be prepped and draped in the standard manner. This will include insertion of an indwelling bladder catheter using standard aseptic technique to allow identification of the urethra and also application of anti-thomboembolic pneumatic sequential compression stockings to the lower limbs. A weighted vaginal retractor or other suitable form of retractor such as the “Lone Star”198 is used.

Two pairs of Allis Forceps, or similar, are then applied, in the sagittal plane about 5 cms apart, to the cystocele. The inferior pair of such forceps is placed proximate to the bladder neck. The intervening vaginal wall of the cystocele is placed on traction between the clamps and infiltrated, using a 22 gauge needle, with an adequate volume of saline containing suitable local anesthetic and vasoconstrictor agents This will facilitate optimal hydrodissection and hemostasis.

While maintaining opposing traction on the Allis Forceps, a small incision with a maximum length of approximately 3 cms is made in the vaginal wall commencing in the region of the bladder neck and proceeding in the midline in a cephalad direction toward the vaginal apex. The use of hydrodissection allows the incision to be deep enough to reach the bladder fascia (pubocervical fascia) in a safe manner and thus minimize failure of wound healing with subsequent mesh extrusion. Initial sharp then blunt dissection technique with the fingertip—well known to workers in the field, is then used to separate the bladder from the anterior vaginal wall and reach and identify in turn, the ischial spine and sacrospinous ligaments on both sides of the pelvis.

The invention is then passed in a closed and circularly folded configuration completely through the vaginal incision in the midline toward the sacrum, between the vagina and the bladder. The device is then allowed to unfold by inherent elastic recoil and digitally positioned into the desired anatomical location previously described. On each side of the pelvis, the posterior frame of the invention will be positioned just above and proximate to the ischial spine and be gently fixed by short projections, incorporated into the polymer frame as previously described into the fibromuscular tissues of the coccygeus muscle. Alternative embodiments and methods of fixation of the posterior component of the frame of the invention into the region of the coccyges muscle include for example biodegradable barbs suitable for fingertip compression.

With regard fixation of the anterior component of the frame on each side of the pelvis into the fibromuscular tissues of the obturator internus muscle, similar short projections incorporated into the frame or biodegradable barbs with fingertip compression may again be utilized.

The short vaginal incision may then be closed using methods familiar to gynecological surgeons. This may include a continuous non-locking suture of 2o or 3o delayed absorbable suture.

Using the embodiments of the present invention, the pelvic repair procedures can be carried out with a minimum of suturing. The frame structure tends to hold the mesh fabric or sheet of graft material in its deployed state and only a few bio-degradable anchor pins of the type currently being used in other surgical hernia repair procedures to secure hernia repair patches in place, will be used to secure the fabric in place should it be desired to remove the frame structure 14 prior to closing the surgically created opening in the vaginal wall.

This invention has been described herein in considerable detail in order to comply with the patent statutes and to provide those skilled in the art with the information needed to apply the novel principles and to construct and use such specialized components as are required. However, it is to be understood that the invention can be carried out by specifically different equipment and devices, and that various modifications, both as to the equipment and operating procedures, can be accomplished without departing from the scope of the invention itself.

What is claimed is:

1. An implantable device for pelvic floor repair comprising:
(a) a thin, flexible sheet having a predetermined shape configuration;
(b) a support frame for maintaining said sheet in its predetermined shape configuration following implantation of said device proximate the pelvic floor of a female patient; and
(c) said support frame being affixed to said sheet and including first and second wing portions that are bilaterally symmetrical about a central axis of the device, the wing portions including rounded wingtip portions at first ends thereof adapted to abut the sacrospinous ligaments when implanted in a female patient, said wingtip portions on the first and second wing portions being integrally joined to one another by a concave, arcuate segment, with end portions of the first and second wing portions opposite the wingtip portions, being dimensioned to rest upon the inferior pubic rami of said female patient when the wingtip portions engage the sacrospinous ligaments.

2. The implantable device as in claim 1 wherein said support frame comprises a plurality of strands of a shape memory material wound together as a cable.

3. The implantable device as in claim 2 wherein the shape memory material comprises Nitinol.

4. The implantable device as in claim 1 wherein the support frame comprises at least one strand of a shape memory material.

5. The implantable device as in claim 4 wherein the shape memory material is a biodegradable polymer.

6. The implantable device as in claim 5 wherein the support frame further includes a cushion layer over a predetermined portion of the first and second wing positions.

7. The implantable device as in claim 6 wherein the cushioning layer is separated from the at least one strand of the shape memory material by an air gap.

8. The implantable device as in claim 6 wherein the cushion layer has radially extending anchoring projections thereon.

9. The implantable device as in claim 1 wherein the end portions of the first and second wing portions opposite from the wingtip portions each being V-shaped, spaced-apart and disconnected from one another.

10. The implantable device as in claim 1 wherein said sheet comprises a synthetic mesh.

11. The implantable device as in claim 10 wherein the synthetic mesh comprises one of polypropylene and PTFE.

12. The implantable device as in claim 1 wherein the sheet is a xenograft material.

13. The implantable device as in claim 9 and further including tubular ferules bonded to end portions of the first and second wing portions to prevent fraying of said cable.

14. The implantable device as in claim 13 wherein said frame includes convex arcuate segments extending between said rounded wingtip portions and said end portions of said first and second wing portions.

15. The implantable device as in claim 1 wherein said sheet and frame can be rolled into a reduced-profile, tubular configuration for delivery through a surgically created opening in the patient's body.
